(19) United States
(12) Patent Application Publication
(10) Pub. No.: US 2020/0015736 A1
(43) Pub. Date: Jan. 16, 2020

(54) PROVIDING FEEDBACK TO A PATIENT PERFORMING AN EXERCISE BASED ON MEASURED PARAMETERS FROM A THERAPY DEVICE

(71) Applicants: Meshal Alhathal, Kearny, NJ (US); Maryam Alkhaldi, Kearny, NJ (US)

(72) Inventors: Meshal Alhathal, Kearny, NJ (US); Maryam Alkhaldi, Kearny, NJ (US)

(21) Appl. No.: 16/027,572

(22) Filed: Jul. 5, 2018

Related U.S. Application Data
(60) Provisional application No. 62/529,346, filed on Jul. 6, 2017.

(51) Int. Cl. A61B 5/00; G09B 19/00; A61B 5/0205; A63B 24/00

(52) U.S. Cl. CPC A61B 5/486; G09B 19/003; A61B 5/021; A61B 5/6804; A63B 24/0062; A61B 5/02055

(57) ABSTRACT

A therapy device may include one or more sensors for measuring metrics. The measured metrics may include at least one of heart rate, range of motion, muscular activity, blood pressure, temperature, sweat or respiration of a patient that correspond to an exercise. The therapy device may include a communication component to provide the measurements from the one or more sensors to a computing device. The communication component may receive feedback from the computing device based on a comparison of the measured metrics from the one or more sensors to a set of target metrics associated with the exercise. The therapy device may include one or more lights to provide visual feedback associated with the exercise based on the received feedback from the computing device.

### RELATED APPLICATION

This application claims the benefit under 35 U.S.C. § 119(e) of U.S. Provisional Application 62/529,346 filed on Jul. 6, 2017.

### TECHNICAL FIELD

Implementations of the disclosure relate to therapy devices, and in particular, to providing feedback to a patient performing an exercise based on measured parameters from a therapy device.

### BACKGROUND

Recently, the healthcare industry has shifted the payment method for therapy from pay-per-service to pay-per-performance, also referred to as “Value-Based Care.” This shift has not only incentivized healthcare professionals to deliver better care, but also encouraged healthcare professionals to record and present evidence-based data to show that a patient is progressing as a result of a treatment plan.

### BRIEF DESCRIPTION OF THE DRAWINGS

The accompanying drawings, which are incorporated herein and form a part of the specification, illustrate the disclosure and, together with the description, further serve to explain the principles of the disclosure and to enable a person skilled in the pertinent art to make and use the disclosure. The disclosure is illustrated by way of examples, and not by way of limitation, in the figures of the accompanying drawings.

FIG. 1 illustrates a therapy device, in accordance with embodiments of the disclosure.

FIG. 2 is a block diagram illustrating an exemplary system architecture, in accordance with embodiments of the disclosure.

FIG. 3 is a flow diagram illustrating a method for performing a calibration session to generate a video including an associated stream of parameters, in accordance with embodiments of the disclosure.

FIG. 4 is a flow diagram illustrating a method for providing feedback based on a stream of parameters received from a therapy device, according to an embodiment of the disclosure.

FIG. 5A illustrates receiving target values associated with an exercise, according to embodiments of the disclosure.

FIG. 5B illustrates providing feedback to a computing device based on the received target values, according to embodiments of the disclosure.

FIG. 5C illustrates providing visual feedback based on different received parameters from a therapy device, according to embodiments of the disclosure.

FIG. 6A illustrates receiving a video corresponding to an exercise that includes a video identification and a first stream of parameters from a storage resource, in accordance with an embodiment of the disclosure.

FIG. 6B illustrates receiving a second stream of parameters from a therapy device during playback of the video, according to some embodiments of the disclosure.

FIG. 6C illustrates storing the second stream of parameters at a storage resource under a video identification, in accordance with embodiments of the disclosure.

FIG. 7A illustrates providing visual feedback based on parameters received from a therapy device during performance of an exercise being outside of a target range, according to embodiments of the disclosure.

FIG. 7B illustrates providing visual feedback based on parameters received from a therapy device during performance of an exercise being within a target range, according to embodiments of the disclosure.

FIG. 8 is a block diagram illustrating an exemplary computer system, according to some embodiments.

### DETAILED DESCRIPTION

Aspects of the disclosure relate to providing feedback to a patient performing an exercise based on measured parameters from a therapy device. The therapy device may be operatively coupled to a computing device and a storage resource via a network. The therapy device may include one or more sensors for measuring metric data associated with a patient's physical activities. The metric data may be compared to acquired metric data and/or a set of a target values and feedback may be provided based on the comparison.

As the healthcare industry transitions to a pay-per-performance method of payment, there is an increased emphasis being placed on the gathering and presentation of evidence-based data in order to show the effectiveness of a treatment plan for a patient. Accordingly, many hospitals and health organizations have been exploring new methods of improving patient documentation and outcomes. Conventional solutions to improve patient documentation and outcomes may be implemented using software, such as an application executed by a mobile device or a computing device. A conventional software application may provide a treatment plan that guides a patient through a series of exercises using step-by-step pictures, games or videos. Upon completion of the exercise, the patient may provide an input to the application indicating that the exercise has been completed. For example, a software application may display a series of pictures illustrating an exercise that is to be performed by the patient. Then, once the patient has completed the exercise, the patient may select an icon within the software application indicating that the patient has completed the exercise.

However, a conventional software application may not receive metric data, such as heart rate, range of motion or muscle activity, from the patient during performance of the exercise. Accordingly, the software application may not be aware of whether or not the patient has performed the exercise properly (e.g., achieved a target range of motion, heart rate, etc.), or if the patient has performed the exercise at all. Furthermore, since no metric data is gathered by the software application, the software application may be unable to determine whether the treatment plan for the patient is effective.

A conventional hardware solution may include sensors to that are attached to a patient to measure metric data as the patient performs an exercise. After attaching the sensors, the patient may perform preconfigured exercises and provide feedback to let the patient know whether or not the patient is performing the exercise properly. However, conventional hardware solutions require the use of preconfigured exercises and may not allow a therapist to create a customized treatment plan to most effectively treat a patient. Additionally, conventional hardware solutions require a sanitization process after each use to sterilize the sensors that are attached directly to the skin, which is a time consuming and tedious process.

Aspects of the disclosure address the above and other deficiencies by providing feedback to a patient performing an exercise based on measured parameters from a therapy device. During an initial treatment session, a therapist may attach a therapy device to a patient. The therapy device may include one or more sensors for measuring the metric data of a patient. The therapy device may further include a communication component, such as a wireless transmitter, to provide metric data to a computing device. Upon attaching the therapy device to the patient, the therapist may initiate a calibration session. During the calibration session, the patient may perform a series of exercises under the instruction of the therapist that correspond to a treatment plan for the patient. As the patient is performing the exercises, a content item of the patient performing the exercises may be recorded. Examples of types of content items include videos, photographs, audio recordings, text and the like. For illustration purposes, embodiments of the disclosure may be described using a video type of content item. At the same time, metric data measured by the one or more sensors of the therapy device during performance of the exercise (also referred to as a “stream of parameters” hereafter) may be transmitted to a computing device for storage. After the patient has finished performing the exercises of the treatment session, the stream of parameters may be assigned one or more timestamps that may be used to align stream of parameters so that the stream of parameters is synchronized with the exercise being performed on the recorded video.

In embodiments of the disclosure, the therapist may use a software application on the computing device to set target values for the initial and/or subsequent performances of the exercises based on the metric data acquired during the calibration session. For example, if a patient achieved a range of motion of 90 degrees for an exercise during the calibration session, then the therapist may set a target value of 90 degrees range of motion for subsequent performances of the exercises. In embodiments, a therapist may set the target value as a portion, such as a percentage, of the stream of parameters. For example, the therapist may set the target value to be 80% of the stream of parameters. Accordingly, if the patient achieved a range of motion of 90 degrees for an exercise during the calibration session, then the target value may be 72 degrees (e.g., 80% of 90 degrees). In another example, if the patient achieved a range of motion of 90 degrees for an exercise during the calibration session, then the target value may be a range of values between 72 and 108 degrees (e.g., 80% and 120% of 90 degrees respectfully). After the target values have been set, the recorded video, the stream of parameters and the target values may be stored in a storage resource, such as a cloud based storage system and/or a local storage, with an associated video identification.

The subsequent performance of the exercises may be performed by the patient at home or at a medical facility. The therapy device may be attached to the patient in a similar location as the therapy device was attached during the calibration session. The patient or therapist may then access the storage resource, such as a cloud based storage system and/or a local storage, using a computing device and locate the video recorded during the calibration session using the video identification. Upon selection of the video, the video as well as the stream of parameters and target value associated with the video may be loaded from the storage resource to the computing device for playback by the computing device.

Upon initiation of the playback of the video, the patient may begin performing the exercises that are being played in the video while wearing the therapy device. During the performance of the exercises in the video, the therapy device may transmit a stream of parameters measured from the sensors of the therapy device to the computing device via the communication component. The computing device may receive the stream of parameters from the therapy device and perform a comparison to the stream of parameters that were obtained during the calibration session to determine if the target value is being met during the performance of the exercise. Based on the comparison, the computing device may transmit a signal to the therapy device that causes the therapy device to provide feedback to the patient that indicated whether or not the patient is meeting the target value during performance of the exercise.

For example, if the patient is meeting the target value during performance of the exercise, the computing device may transmit a signal to the therapy device that causes a light on the therapy device to light up green. Alternatively, if the patient is not meeting the target value during the performance of the exercise, the computing device may transmit a signal to the therapy device that causes a light on the therapy device to light up red. Upon completion of the subsequent performance of the exercise, the new stream of parameters may be stored at the storage resource with the original stream of parameters under the video identification.

Thus, aspects of the disclosure may be advantageous by allowing a therapist to create a customized treatment plan for a patient rather than requiring a therapist to choose from a set of predefined exercises. Furthermore, the ability to provide feedback to the patient during performance of subsequent performances of the exercise helps the patient determine whether or not he or she is performing the exercise correctly. Additionally, the storage of the streams of parameters under the video identification at the storage resource allows the therapist to easily access a patient's metric data to determine how effective the treatment plan is for the patient. Aspects of the disclosure improve the ease of user interactions with the system by providing a customized treatment plan to a user and providing feedback in real time to the patient during the performance of the exercises in the treatment plan.

Although embodiments of the disclosure are described in the context of a physical therapy treatment plan, aspects of the disclosure may be applied to any sort of exercise or physical fitness plan. For example, a first user may record a workout routine while wearing the therapy device. A stream of parameters may be received from the therapy device during performance of the exercise by the first user. Then, a second user may perform the exercise in the recorded video of the first user while wearing a therapy device and try to match the stream of parameters that corresponds to the first user's performance of the exercise.

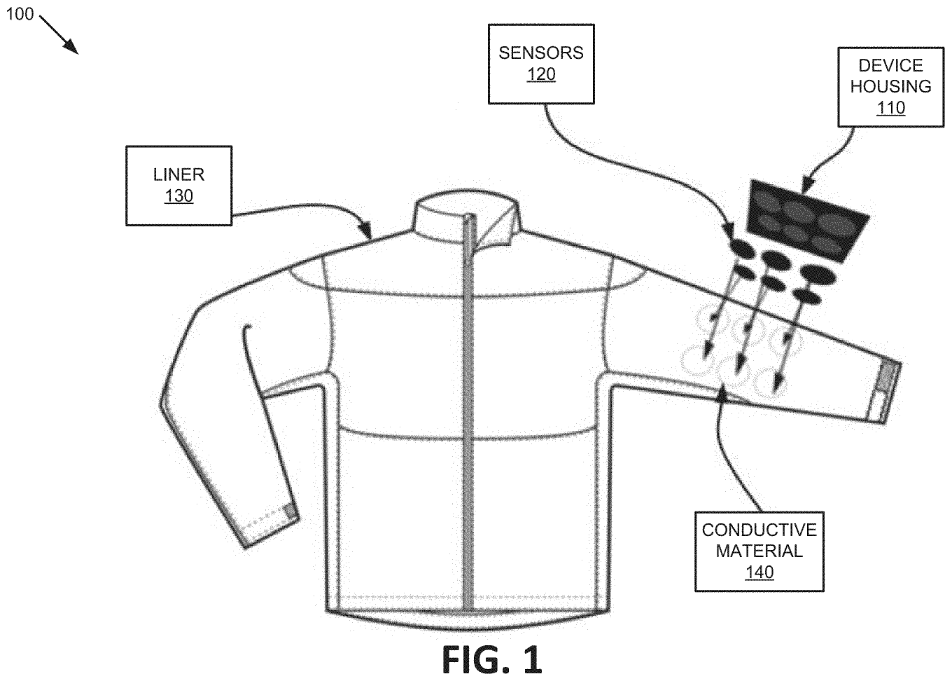

FIG. 1 illustrates a therapy device 100, in accordance with embodiments of the disclosure. The therapy device 100 may include a device housing 110. The device housing 110 may provide a housing for circuitry, such as a communication component (not shown), and lights (not shown) of the therapy device. In an embodiment, the device housing 110 may be composed of a waterproof or water resistant material in order to prevent exposure of the circuitry to moisture. In embodiments, the device housing 110 may include one or more straps to hold the device housing 110 in place on a patient.

One or more sensors 120 may be disposed on a bottom surface of the device housing 110. The one or more sensors 120 may measure metric data of the patient. Examples of metric data measured by the one or more sensors include, but are not limited to, heart rate, range of motion, muscle activity and blood pressure. In an embodiment, the one or more sensors 120 may be separate from the device housing 110 and may be coupled to the device housing 110 via cables or wireless transmitters.

In one embodiment, the one or more sensors 120 may include one or more electrocardiogram (ECG) sensors to acquire heart rate measurements. The ECG sensor measures the electrical activity in muscle depolarizations of the heart. In embodiments, the ECG sensor may include 12-lead electrodes that are gel-based, which are placed on the patient's upper chest and arms. In an embodiment, the ECG sensor may be a one-lead ECG sensor that includes a single, gel-based electrode placed on the patient. In embodiments, the one or more sensors 120 may include a photoplethysmography (PPG) sensor to acquire heart rate measurements. A PPG sensor is placed on the skin of a patient and uses a pulse oximeter to illuminate the skin of the patient and measure changes in light absorption. The PPG sensor may then determine the heart rate of the patient based on the measured change in light absorption.

In embodiments, the one or more sensors 120 may include one or more electromyography (EMG) sensors to measure muscle activity. The EMG sensor uses one or more electrodes to detect electrical signals from motor neurons that cause muscles to contract. Metric data corresponding to muscle activity is determined based on the received electrical signals from the one or more electrodes.

In embodiments, the one or more sensors 120 may include one or more inertia measurement units (IMUs) for measuring range of motion. In embodiments, the IMU may be a nine-degree-of-freedom IMU that includes a triple-axis accelerometer, a triple-axis gyroscope and a triple-axis magnetometer.

In an embodiment, the one or more sensors 120 may include, but are not limited to, cuff or cuffless Sphygmomanometer (e.g., blood pressure) sensors, temperature sensors, sweat sensors, and/or respiratory sensors.

In an embodiment, the therapy device 100 may include a liner 130 that can be worn by the patient. The liner 130 may be worn by the patient to prevent the one or more sensors 120 from being in direct contact with the patient's skin, reducing or eliminating the need of sanitization of the one or more sensors 120 after use of the therapy device. The liner 130 may include one or more portions that are made of a conductive material 140. The one or more portions that are made of the conductive material 140 may correspond to locations for placement of the one or more sensors 120. The conductive material 140 can pass biosignal voltages between the body of the patient and the one or more sensors 120. In some embodiments, the conductive material 140 may be combined with or replaced by a non-conductive material with embedded electrodes. In embodiments, the liner 130 may be made of a disposable material so that the liner 130 may be disposed of after use. In an embodiment, the liner 130 may be made of a reusable material, such as a cloth, that may be washed and reused between treatment sessions. In some embodiments, the one or more sensors 120 may be placed directly on the skin of the patient without use of the liner 130.

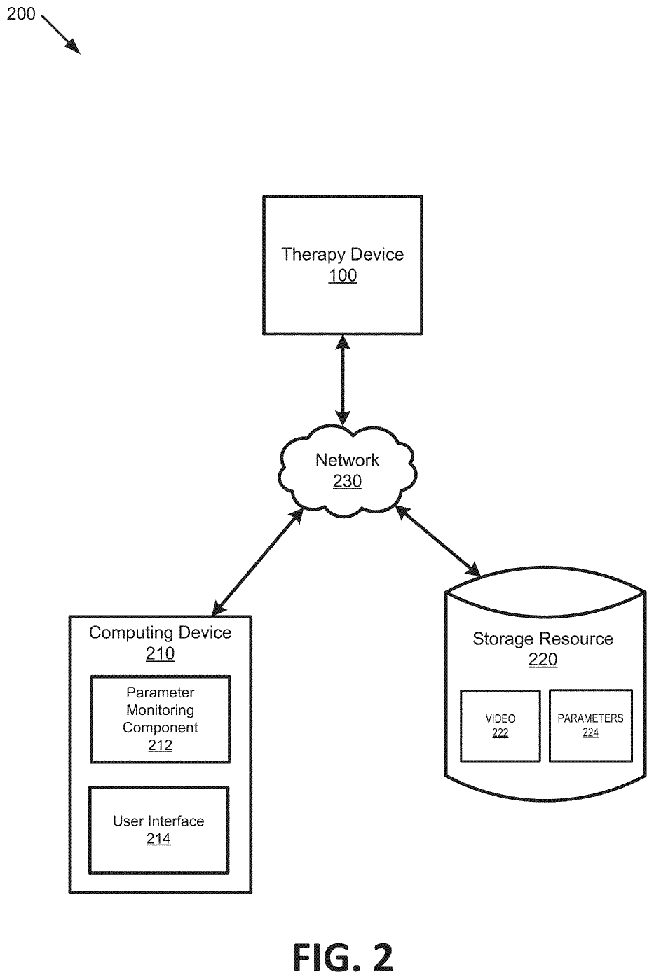

FIG. 2 illustrates an example system architecture 200 for implementing a physical therapy treatment system, in accordance with an embodiment of the disclosure. The system architecture 200 includes a therapy device 100, a computing device 210, and a storage resource 220. The components 100, 210 and 220 may be communicatively coupled/connected via a network 230. In one embodiment, the network 230 may include a public network (e.g., the Internet), a private network (e.g., a local area network (LAN) or wide area network (WAN)), a wired network (e.g., Ethernet network), a wireless network (e.g., an 802.11 network, Bluetooth, NFC, or a Wi-Fi network), a cellular network (e.g., a Long Term Evolution (LTE) network), routers, hubs, switches, server computers, and/or a combination thereof. In some embodiments, the network 230 may be a combination of different types of networks. In embodiments, components 100, 210 and 220 may be coupled via physical connections, such as a Universal Serial Bus (USB) or serial cable.

In an embodiment, the storage resource 220 may be a memory (e.g., random access memory), a cache, a drive (e.g., a hard drive), a flash drive, a database system, or another type of component or device capable of storing data. The storage resource 220 may also include multiple storage components (e.g., multiple drives or multiple databases) that may also span multiple computing devices (e.g., multiple server computers), and may be cloud-based. In some embodiments, the storage resource 220 may be a part of the computing device 210. In some embodiments, the storage resource 220 may be distributed among and accessible to the computing device 210 and the therapy device 100. One or more of the devices of the system architecture 200 may utilize the storage resource 220 to store public and private data, such as videos 222 corresponding to exercises of a treatment plan and parameters 224 associated with the videos 222. The storage resource 220 may be configured to provide secure storage for private data.

In one embodiment, the computing device 210 may include computing devices such as personal computers (PCs), laptops, mobile phones, smart phones, tablet computers, netbook computers, etc. Although a single computing device 210 is illustrated in FIG. 2, more than one computing device 210 may be part of system architecture 200. An individual user may be associated with (e.g., own and/or use) the computing device 210. As used herein, a “user” may refer generally to an individual operator of the computing device 210, such as a therapist or patient.

In one implementation, the computing device 210 may implement a user interface 214. The user interface 214, which may also be referred to as a graphical user interface (GUI), may allow a user of the respective computing device 210 to send/receive information to/from the therapy device 100 and/or the storage resource 220. For example, the user interface 214 may be a web browser interface that can access, retrieve, present, and/or navigate data (e.g., video 222 and parameters 224) provided by the storage resource 220. In one implementation, the user interface 214 may be a standalone dual environment application (e.g., a mobile app). The user interface 214 may allow for the computing device 210 to send/receive information to/from the therapy device 100 and/or the storage resource 220.

In one implementation, a parameter monitoring component 212 executes on the computing device 210. The parameter monitoring component 212 may receive a video of an exercise and retrieve a first stream of parameters including one or more timestamps for the video. The parameter monitoring component 212 may align the video with the first stream of parameters using the one or more timestamps. The parameter monitoring component 212 may determine a target value associated with at least one parameter of a stream of parameters during performance of the exercise. The parameter monitoring component 212 may receive a second stream of parameters from a therapy device, such as therapy device 100, attached to a patient performing the exercise in the video. The parameter monitoring component 212 may determine that the parameter satisfies the target value and provide feedback indicating that the parameter satisfies the target value. Further details with regards to the parameter monitoring component 212 are provided below.

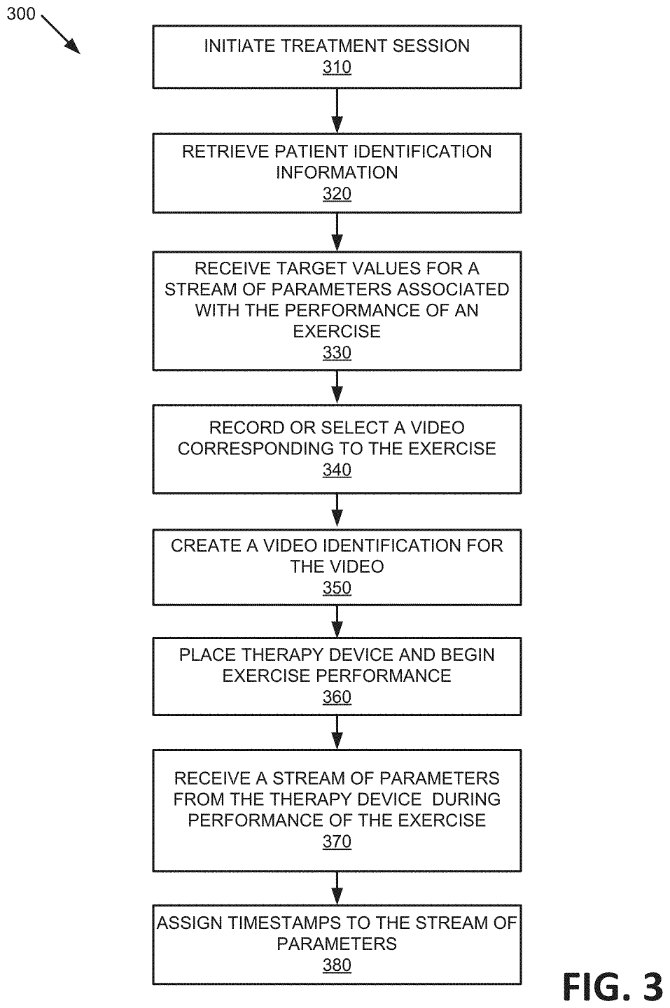

FIG. 3 is a flow diagram illustrating a method for performing a calibration session to generate a video including an associated stream of parameters, in accordance with an embodiment of the disclosure. The method 300 may be performed by processing logic that comprises hardware (e.g., circuitry, dedicated logic, programmable logic, microcode, etc.), software (e.g., instructions run on a processing device to perform hardware simulation), or a combination thereof.

For simplicity of explanation, the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events. Additionally, it should be appreciated that the methods disclosed in this specification are capable of being stored on an article of manufacture to facilitate transporting and transferring such methods to computing devices. The term “article of manufacture,” as used herein, is intended to encompass a computer program accessible from any computer-readable device or storage media. In one implementation, method 300 may be performed by various components of system architecture 200 as shown in FIG. 2.

At block 310, a treatment session is initialized. In an embodiment, the treatment session may be a calibration session to create an exercise video for a treatment plan, a stream of parameters and one or more target values for a new patient. In embodiments, the treatment session may be initialized by launching a software application on a computing device, such as computing device 210 of FIG. 2. The software application may include a GUI, such as user interface 214 of FIG. 2, which may include one or more selectable icons to facilitate various actions by the software application. For example, the GUI may include a selectable icon that, when selected, initiates a treatment session including one or more exercises.

At block 320, patient identification information is retrieved. If the patient is an existing patient, the patient identification information may be retrieved from a storage resource, such as a cloud based storage system. In embodiments, the patient identification information may be accessed via the GUI of the software application. If the patient is a new patient, then the patient identification information may be entered for storage at the storage resource. In embodiments, the patient identification information may be entered via the GUI of the software application. For example, the GUI of the software application may provide a form with fields for patient identification information (e.g., name, birthdate, etc.), which can be filled out and submitted for storage at the storage resource.

At block 330, one or more target values for a stream of parameters associated with the performance of an exercise are received. The one or more target values may be entered by a therapist after performing an initial assessment of a patient. The one or more target values may correspond to a particular parameter of the stream of parameters. For example, a received target value for range of motion may be set at 90 degrees. In an embodiment, the one or more target values may be received via the GUI of the software application. For example, a therapist may enter the one or more target values via one or more fields in the GUI of the software application. In embodiments, the one or more target values may correspond to a threshold value of a parameter. In some embodiments, the one or more target values may correspond to a maximum and minimum value of a parameter. For example, the received target values of range of motion may correspond to a minimum value of 80 degrees and a maximum value of 110 degrees. In embodiments, multiple target values may be assigned to different times and/or segments of the video. For example, a first target value may be assigned to seconds 0-10 of a video and a second target value may be assigned to seconds 10.1-20 of the video.

At block 340, a video that corresponds to an exercise is recorded or selected. In embodiments, the video may be a recording of the patient performing the exercise during the calibration session, as discussed in further detail below at block 360. In an embodiment, the video may be a previously recorded video of the exercise that may be selected from a library of exercise videos stored at a storage resource. In some embodiments, the video may be constructed of various previously recorded segments that are combined to form an exercise video. For example, the video may be constructed of five previously recorded 10 second video clips, which are combined to create the exercise video.

In an embodiment, the video that corresponds to the exercise may be recorded or selected via the GUI of the software application executing on the computing device. For example, a user (e.g., a therapist or patient) may select an icon within the GUI of the software application to record a new video. Upon selection of the icon, the software application may access a camera or recording device operatively coupled to the computing device to record the video of the patient performing the exercise. The GUI of the software application may provide a recording interface to facilitate the recording of the video. The recording interface may include one or more selectable icons to begin recording the video. The recording interface may further include a video frame to display a current view of the camera or recording device.

In embodiments, a user may select an icon within the GUI of the software application to select a previously recorded exercise video. The GUI of the software application may provide a listing of one or more previously recorded exercise videos that are available for selection. In embodiments, one or more of the previously recorded exercise videos may be selected for use during the calibration session.

In an embodiment, a user may select an icon within the GUI of the software application to select one or more previously recorded exercise segments. The GUI of the software application may provide a listing of one or more previously recorded exercise segments that are available for selection. One or more of the previously recorded exercise segments may be selected for use during the calibration session. The GUI may provide an interface for the arrangement of the one or more exercise segments in a particular order. For example, if exercise segments A, B and C are selected via the GUI, the interface may allow the segments to be arranged such that exercise segment B is first, exercise segment C is second and exercise segment B is third. The one or more exercise segments may then be stored at a storage resource in the designated order for subsequent playback.

At block 350, a video identification is created for the video. The video identification may correspond to a name or number associated with the video. In embodiments, the video and the target values received at block 330 may be stored at a storage resource under the video identification. The video identification may be used to identify and retrieve the video and any associated target values and/or streams of parameters associated with the video. In embodiments, the video identification may be created after the recording of the video or the calibration session has completed.

At block 360, a therapy device, such as therapy device 100 of FIG. 1, is placed on the patient. Once the therapy device has been placed on the patient, the patient may begin performing the exercise. In an embodiment, a camera or recording device may record a video of the patient as the patient performs the exercise. In another embodiment, playback of a previously recorded exercise video or one or more previously recorded video segments may be initiated. For example, a previously recorded exercise video may begin playback on a computing device.

At block 370, a stream of parameters is received from the therapy device during performance of the exercise. In one embodiment, the patient may perform the exercise based on received instructions from the therapist while being recorded. In another embodiment, the patient may perform the exercise shown in the previously recorded exercise video being played back on the computing device. The stream of parameters may be received by the computing device from the therapy device via a communication component, such as a wireless transmitter, of the therapy device. The stream of parameters may correspond to metric data measured from the one or more sensors of the therapy device during performance of the exercise. In some embodiments, the stream of parameters may not be provided by the therapy device until completion of the exercise. For example, the stream of parameters may be stored on a memory of the therapy device and transmitted to the computing device once the exercise has been completed. Upon completion of the exercise, if the patient's performance of the exercise is being recorded, the recording of the video may be stopped via a recording interface on the computing device.

At block 380, one or more timestamps are assigned to the stream of parameters received at block 370. The one or more timestamps may serve as reference points in the stream of parameters that correspond to particular segments of the exercise. For example, a timestamp may be assigned to a particular time in the stream of parameters that indicates the beginning of a particular segment of the exercise. The timestamps may be used to align the stream of parameters for an exercise with a video of the exercise, as will be described in further detail below. Upon assigning the one or more timestamps to the stream of parameters, the calibration session is complete. After the calibration session has completed, the video, the target values and the stream of parameters with assigned timestamps may be stored at a storage resource.

In some embodiments, the timestamps may be standard timestamps where each timestamp has an assigned time and date. In embodiments, the timestamps may correspond to a number of attributes in one or more entities that are recorded in a uniform fashion. For example, recording a ten-minute exercise video with sensors that update each second, the first five attributes may correspond to the first five seconds of the video. In another example, the last minute of the video may correspond to the last sixty attributes of the entities. In embodiments, the video, the target values and the stream of parameters with assigned timestamps may be stored at the storage resource under the video identification created at block 350.

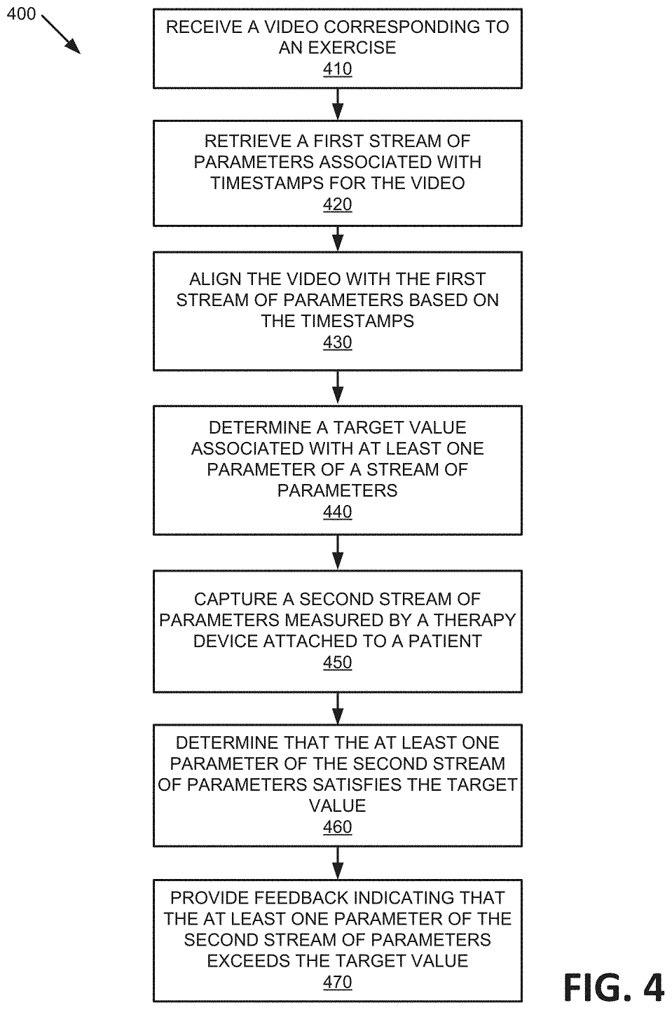

FIG. 4 is a flow diagram illustrating a method for performing a calibration session to generate a video including an associated stream of parameters, in accordance with an embodiment of the disclosure. The method 400 may be performed by processing logic that comprises hardware (e.g., circuitry, dedicated logic, programmable logic, microcode, etc.), software (e.g., instructions run on a processing device to perform hardware simulation), or a combination thereof. In one implementation, method 400 may be performed by various components of system architecture 200 as shown in FIG. 2.

At block 410, a video that includes an exercise is received. In an embodiment, the video may be received from a cloud based storage system. In embodiments, the video may be received from a local database. In an embodiment, the video may be received from a memory device, such as a flash drive or external hard drive. In embodiments, the video may include a video identification, such as a title or number, used to identify the video. In some embodiments, the video may be a video created during a calibration session, as previously described in FIG. 3.

At block 420, a first stream of parameters for the video is retrieved from a storage resource. The first stream of parameters may include one or more timestamps. In embodiments, the first stream of parameters and timestamps may be created during a calibration session, as previously described in FIG. 3. In an embodiment, the first stream of parameters may be retrieved from the storage resource using the video identification of the video.

At block 430, the video may be aligned with the first stream of parameters based on the timestamps. As previously discussed, the timestamps of the first stream of parameters may serve as reference points to particular segments of the exercise being performed in the video. The timestamps may be used to align the first stream of parameters with the exercise being performed in the video, such that the timestamp of the first stream of parameters for a particular segment of the exercise is at a same or substantially similar time as when the particular segment of the exercise is taking place during the playback of the video. By aligning the first stream of parameters with the exercise being played in the video, a comparison can be made between the first stream of parameters and another stream of parameters received during a subsequent performance of the exercise by a patient as the patient performs the exercise during playback of the video. For example, a comparison can be made between the first stream of parameters that were acquired during a calibration session under the instruction of a therapist and another stream of parameters received during performance of the exercise by the patient at home while watching the exercise video.

At block 440, a target value associated with at least one parameter of the stream of parameters is determined. In an embodiment, the target value may correspond to the target value of the calibration session, as described in FIG. 3. In embodiments, the target value may correspond to a threshold value for a parameter of the stream of parameters received during performance of the exercise. For example, the target value may be a threshold of 90 degrees range of motion during performance of the exercise by a patient. In an embodiment, the target value may be based on the first stream of parameters. For example, the target value may be a percentage of the first stream of parameters. In some embodiments, the target value may be a maximum and/or minimum value for a parameter of the stream of parameters. For example, the target value may be a minimum range of motion of 80 degrees and a maximum range of motion of 110 degrees.

In embodiments, target values for an exercise may be modified. For example, a patient and/or therapist may find that a target value is set too high and is unattainable by the patient during performance of the exercise. Conversely, as a patient progresses through a treatment plan, the patient and/or therapist may find that the target value is set too low and is too easy to meet by the patient during performance of the exercise. In an embodiment, the target value may be modified via a GUI of a software application executed by a computing device. For example, the therapist may access the software application and enter a new target value for a particular video in the GUI. The new target value may then be stored at the storage resource under the video identification of the particular video for subsequent use with the video.

In an embodiment, a machine learning model executed by a computing device may be used to identify and modify target values for the stream of parameters. The machine learning model may receive streams of parameters for a patient performing a particular exercise of a video. Over time, the machine learning model may identify that one or more target values for a stream of parameters should be modified based on the patient either consistently meeting or not meeting the target value. Upon identifying the one or more target values, the machine learning model may determine new target values based on the received streams of parameters. For example, a target value may be set at 90 degrees, but the patient may only be able to achieve a range of motion between 75 and 82 degrees during performance of an exercise. Accordingly, the determined new target value may be set at 80 degrees, which may be considered challenging by the patient, but still attainable by the patient.

At block 450, a second stream of parameters measured by a therapy device attached to a patient is received. The second stream of parameters may be received by a computing device from the therapy device via a communication component. The second stream of parameters may correspond to metric data measured by the one or more sensors of the therapy device during performance of the exercise of the video by the patient.

At block 460, at least one parameter of the second stream of parameters is determined to satisfy the target value. For example, if the target value for a particular segment of an exercise is a minimum of 90 degrees range of motion and the measured parameter is 92 degrees range of motion during the particular segment of the exercise, then the parameter satisfies the target value. In another example, if the target value for the particular segment is a minimum of 75 degrees range of motion and a maximum of 90 degrees range of motion and the measured parameter is 83 degrees range of motion, then the parameter satisfies the target value.

In embodiments, the target value may correspond to a comparison between the first stream of parameters and the second stream of parameters that update periodically. For example, the target value may update each second to guide a patient to replicate the exercise patterns as the exercise patterns were performed under therapist supervision.

At block 470, feedback is provided that indicates the at least one parameter of the second stream of parameters satisfies the target value. In an embodiment, the feedback may be visual feedback. For example, the computing device may transmit a signal to the therapy device that causes one or more lights on the therapy device to light up, notifying the patient wearing the therapy device that the received parameters during performance of the exercise satisfy the target value. In another example, the computing device may provide visual feedback in a GUI of a software application indicating whether the parameters during performance of an exercise satisfy the target value, as will be discussed in more detail below.

In some embodiments, the feedback may be audio feedback. For example, if the parameters during performance of the exercise satisfy the target value, the computing device may provide an audible statement such as “Good job!” audibly indicating to the patient that the parameters satisfy the target value. In embodiments, the feedback may be haptic feedback. For example, if the parameters during performance of the exercise satisfy the target value, a vibrating motor coupled to the therapy device may vibrate to indicate to the patient that the parameters satisfy the target value. In embodiments, other forms of feedback may be provided to the patient that indicate to the patient that parameters during the performance of an exercise satisfy the target value.

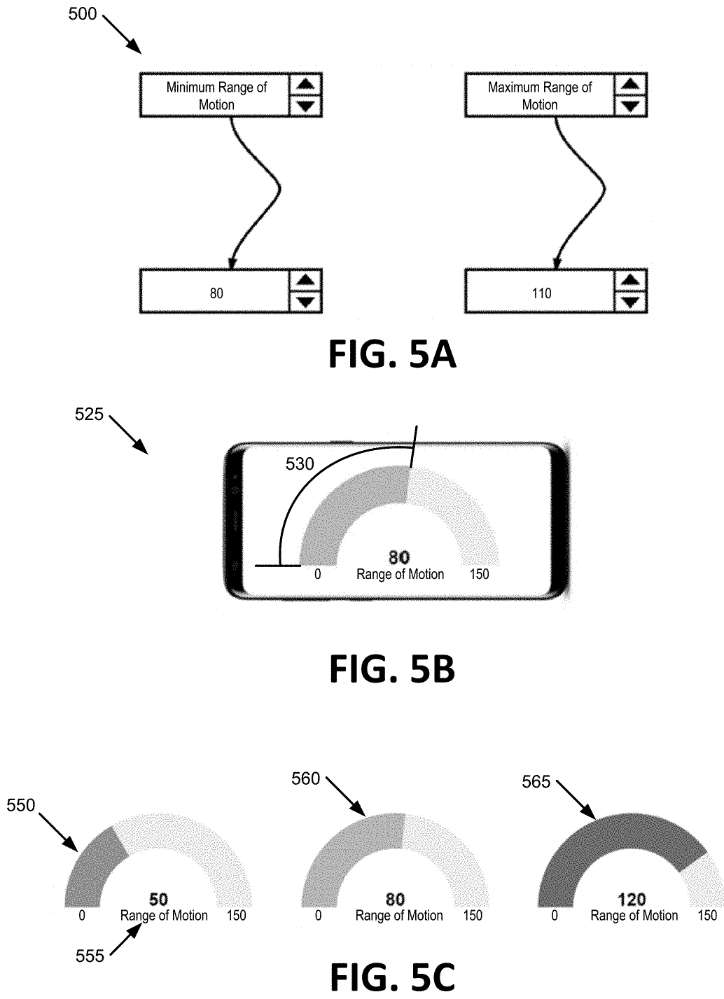

FIG. 5A illustrates receiving target values 500 associated with an exercise, according to embodiments of the disclosure. In an embodiment, the target values 500 may correspond to the received target values at block 330 of FIG. 3. In embodiments, the target values 500 may correspond to target values that modify the target values received at block 330. The target values 500 may be entered via a GUI on a computing device. In an embodiment, a user of the computing device (e.g., a therapist or patient) may be provided with one or more fields in the GUI to enter target values for an exercise. In the illustrated example, a user of the computing device has entered a target value corresponding to a minimum and maximum range of motion in the fields of the GUI. The minimum range of motion has been set at 80 degrees range of motion and the maximum range of motion has been set at 110 degrees.

FIG. 5B illustrates providing feedback to a computing device 525 based on the received target values, according to embodiments of the disclosure. In the illustration, the computing device may correspond to a mobile device of a user, such as a patient or therapist. The mobile device may receive a stream of parameters from a therapy device attached to a patient during performance of an exercise and provide visual feedback to indicate whether one or more parameters of the stream of parameters satisfy the target values for the exercise. The visual feedback may correspond to a current value of the one or more parameters of the stream of parameters measured by a therapy device attached to a patient during performance of the exercise. In the illustration, the visual feedback is a graphical representation in the form of a semi-circle for the one or more parameters associated with the target value within a GUI that is presented on the mobile device. For example, the visual feedback on the mobile device may be a graphical representation of the range of motion measured by the therapy device during performance of an exercise.

In embodiments, visual feedback may be provided within the GUI for multiple parameters. For example, the visual feedback provided within the GUI may include a first graphical representation corresponding to the range of motion of a stream of parameters and a second graphical representation corresponding to muscle activity. In an embodiment, the visual feedback may correspond to an aggregate value of multiple parameters of the stream of parameters. For example, the visual feedback may include a graphical representation of an aggregate value of the range of motion and muscle activity of a stream of parameters.

In the illustration, the semi-circular graphical representation includes a shaded portion 530 that increases or decreases in size based on the measured value of a parameter. For example, if the semi-circle represents a range of 0 to 150 degrees for the range of motion of a stream of parameters, and the current range of motion is 80, then the shaded portion 530 may correspond to 53% (e.g., 80/150) of the semi-circle. In an embodiment, the shaded portion 530 of the semi-circle may increase or decrease in size in real time based on the measured parameter increasing or decreasing during performance of the exercise.

In embodiments, the shaded portion 530 may represent the comparison of the first stream of parameters and the second stream of parameters as discussed at block 460 of FIG. 4. The first stream of parameters that were acquired during a calibration session under the instruction of a therapist may be compared to a second stream of parameters received during the performance of the exercise by the patient at home and/or in clinic while watching the exercise video. If the second stream of parameters received during the subsequent performance of the exercise satisfy a target value, such as a percentage, when compared to the first stream of parameters, the shaded portion 530 may light green. In embodiments, the acceptable range may be assigned by the therapist or patient in the GUI of a computing device, or may be systemically assigned based on the past performances of the patient.

In embodiments, the shaded portion 530 of the semi-circle may display different colors based on whether the received parameter satisfies an associated target value. For example, if the received parameter is less than a minimum target value, the shaded portion 530 may be yellow. If the received parameter is greater than a maximum target value, the shaded portion 530 may be red. If the received parameter satisfies the target value, the shaded portion 530 may be green. In some embodiments, if the patient is failing to achieve a second stream of parameters that is not substantially similar to the first stream of parameters received during a calibration session, a red or yellow color may be assigned in the shaded portion 530 to alert the patient to improve her/his efforts. Although aspects of the disclosure describe the shaded portion 530 as being colored green, yellow or red, other embodiments of the disclosure may use any combination of different colors.

FIG. 5C illustrates providing visual feedback 550 based on different received parameters from a therapy device, according to embodiments of the disclosure. The visual feedback 550 may represent different graphical representations 555, 560, 565 presented on a computing device based on the measured value of the one or more parameters of the stream of parameters that are received from a therapy device attached to a patient during performance of the exercise.

In the illustration, the target value for range of motion is a minimum value of 80 degrees and a maximum value of 110 degrees and the semi-circle represents a range of 0 to 150 degrees for the range of motion of a stream of parameters. Graphical representation 555 may correspond to a received range of motion of 50 degrees from the therapy device. The shaded portion of graphical representation 555 may correspond to 33% (e.g., 50/150) of the semi-circle. In embodiments, since 50 degrees range of motion is less than the minimum target value of 80 degrees, the shaded portion of graphical representation 555 may be yellow. Graphical representation 560 may correspond to a received range of motion of 80 degrees from the therapy device. The shaded portion of graphical representation 560 may correspond to 53% (e.g., 80/150) of the semi-circle. In embodiments, since 80 degrees range of motion satisfies the target value (e.g., is within the minimum value of 80 degrees and the maximum value of 110 degrees), the shaded portion of graphical representation 560 may be green. Graphical representation 565 may correspond to a received range of motion of 120 degrees from the therapy device. The shaded portion of graphical representation 565 may correspond to 80% (e.g., 120/150) of the semi-circle. In embodiments, since 120 degrees range of motion is greater than the maximum target value of 110 degrees, the shaded portion of graphical representation 565 may be red.

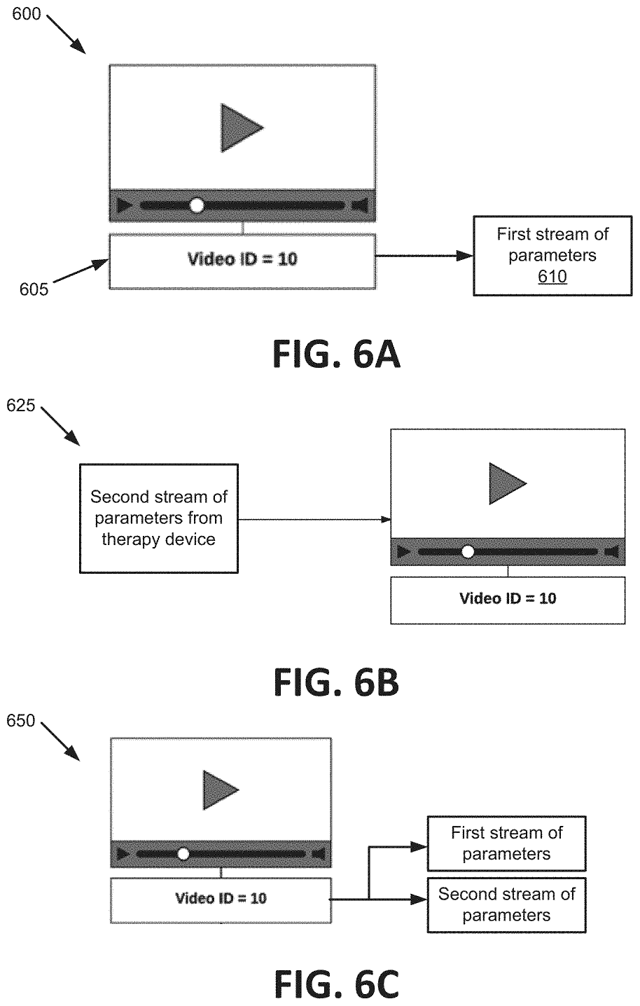

FIG. 6A illustrates receiving a video 600 corresponding to an exercise that includes a video identification and a first stream of parameters from a storage resource, in accordance with an embodiment of the disclosure. In embodiments, the video 600 may be received by a computing device from a storage resource, such as a cloud based storage system. The video 600 may include a video identification 605 and a first stream of parameters 610. In some embodiments, the video 600 may initially include a video identification 605 and upon performing the exercise, the first stream of parameters 610 can be stored at a storage resource under video identification 605. In an embodiment, the video 600, the video identification 605 and the first stream of parameters 610 may be generated during a calibration session, as previously discussed in FIG. 3.

FIG. 6B illustrates receiving a second stream of parameters 625 from a therapy device during playback of the video, according to some embodiments of the disclosure. Upon receiving the video, playback of the video including an exercise may be initiated and the patient may begin performing the exercise. In embodiments, the video may be played back within a GUI on a computing device or mobile device. As the patient performed the exercise, the second stream of parameters 625 may be received from a therapy device attached to the patient. The second stream of parameters may be provided to the computing device by the therapy device via a communication component, such as a wireless transmitter. In some embodiments, the second stream of parameters may be stored locally on a memory of the therapy device and subsequently provided to the computing device upon completion of the exercise.

FIG. 6C illustrates storing the second stream of parameters 650 at a storage resource under a video identification, in accordance with embodiments of the disclosure. Upon completion of the exercise by the patient and receipt of the second stream of parameters by the computing device, the computing device may store the second stream of parameters under the video identification associated with the video at a storage resource. Accordingly, the first stream of parameters, the second stream of parameters and other streams of parameters for subsequent exercises may be located on the storage resource using the same video identification, allowing therapists or other health professionals to easily track a patient's progress and the effectiveness of a treatment plan.

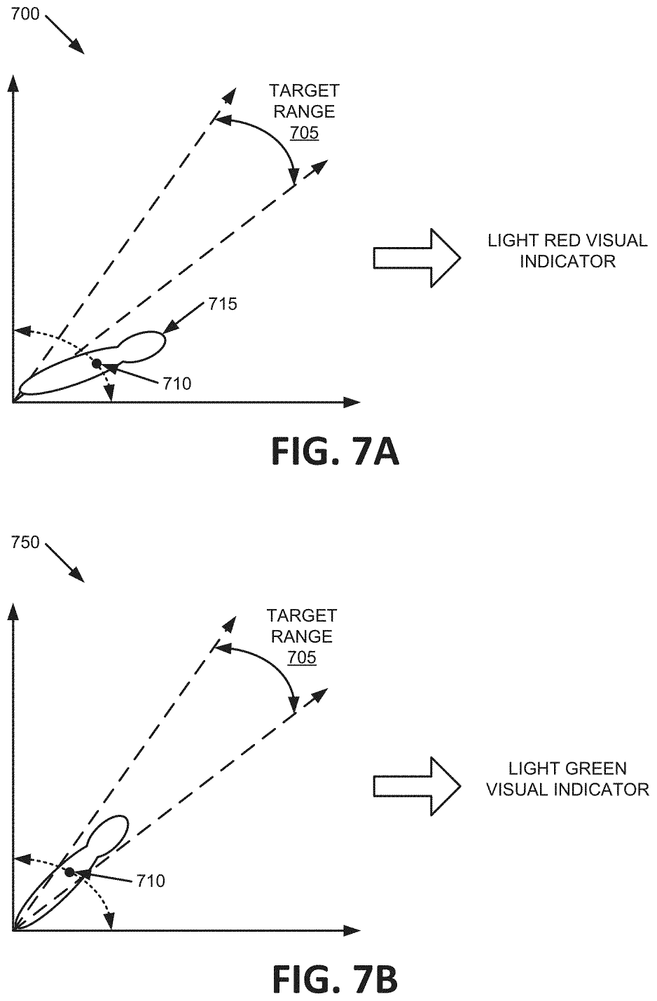

FIG. 7A illustrates providing visual feedback 700 based on parameters received from a therapy device during performance of an exercise being outside of a target range, according to embodiments of the disclosure. A therapy device 710 including one or more sensors may be attached to a patient 715 prior to the performance of an exercise. The exercise may have an associated target range 705 that corresponds to a minimum value and maximum value for the range of motion parameter during performance of the exercise. In some embodiments, the target range 705 may correspond to the comparison results between a first stream of parameters from the performance of an exercise by the patient under the therapist instruction and a second stream of parameters from a subsequent performance of the exercise by at home and/or in clinic. In the illustration, the patient 715 and the therapy device 710 are not within the target range 705 for the range of motion. The therapy device 710 may provide a stream of parameters, including the range of motion, to a computing device. The computing device may receive the stream of parameters and determine that the range of motion is not within the target range 705. Upon determining that the range of motion is not within the target range 705, the computing device may provide a signal to the therapy device 710 that causes one or more lights on the therapy device 710 to light up red. The red light may provide a visual indication to the patient during performance of the exercise that the patient is not currently within the target range. In embodiments, one or more lights for providing a visual indication may not be located on therapy device 710. For example, one or more lights may be operatively coupled to the computing device to provide visual feedback.

FIG. 7B illustrates providing visual feedback 750 based on parameters received from a therapy device during performance of an exercise being within a target range, according to embodiments of the disclosure. In the illustration, the patient 715 and the therapy device 710 are within the target range 705 for the range of motion. The therapy device 710 may provide a stream of parameters, including the range of motion, to the computing device. The computing device may receive the stream of parameters and determine that the range of motion is within the target range 705. Upon determining that the range of motion is within the target range 705, the computing device may provide a signal to the therapy device 710 that causes one or more lights on the therapy device 710 to light up green. The green light may provide a visual indication to the patient during performance of the exercise that the patient is currently within the target range.

In some embodiments, multiple lights may be used to provide visual feedback for different parameters of a received stream of parameters. For example, one light may be used to provide visual feedback corresponding to range of motion, another light may be used to provide visual feedback corresponding to muscle activity and another light may be used to provide visual feedback corresponding to heart rate.

In an embodiment, a switching device may be operatively coupled to the one or more lights that provide visual feedback. The switching device may allow for a selection of providing visual feedback for a particular parameter of the stream of parameters. For example, the switching device may allow for the selection of providing visual feedback for one of range of motion, muscle activity or heart rate. Once the particular parameter has been selected, the one or more lights may provide feedback for the particular parameter that was selected. For example, if muscle activity is selected as the particular parameter, then the one or more lights may provide visual feedback for muscle activity during performance of the exercise. In embodiments, the switching device may include a toggle or rotary switch coupled to the therapy device. In an embodiment, the switching device may allow a selection of feedback for multiple parameters simultaneously. For example, one light may represent muscle activity parameter, another light may represent range of motion, and/or another light may represent heart rate. In an embodiment, the switching device may include a relay.

In some embodiments, the switching device may be implemented via software and/or hardware on a computing device and/or a therapy device. In embodiments, the switching device may include a mechanically-trigged switch, and/or a touch-controlled switch, also known as a capacitive switch. In some embodiments, the switching device may control the type of visual feedback presented on a computing device. In an embodiment, the switching device may control the type of visual feedback presented on the therapy device. In some embodiments, the switching device may be in the form of a GUI such as a toggle switch, checkbox and/or radio presented on a computing device to enable a selection event. The selection event may control the one or more lights on the therapy device and/or the type of visual feedback presented on a computing device.

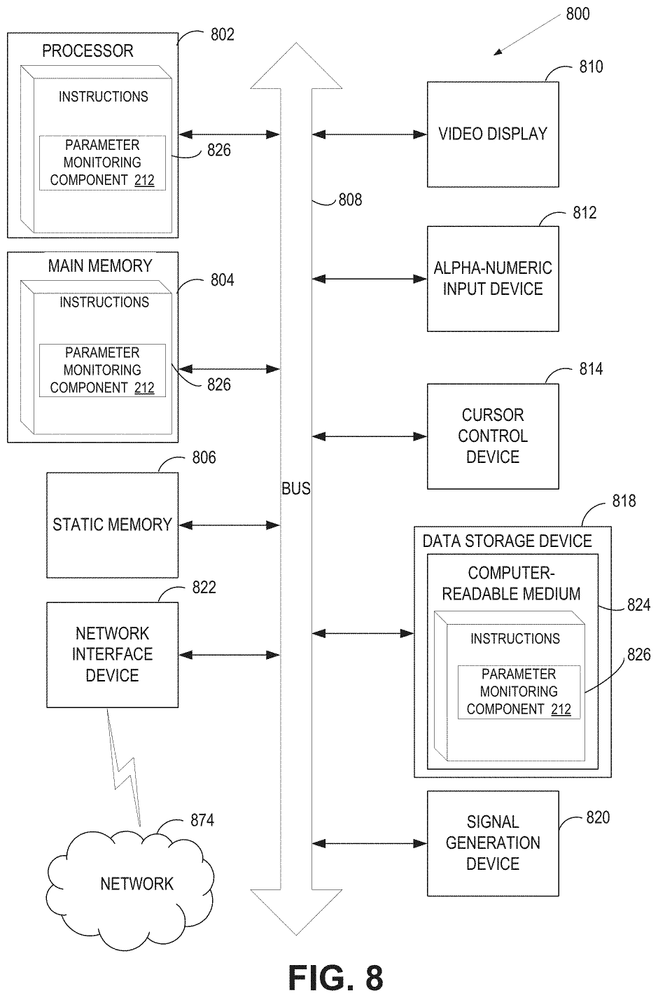

FIG. 8 illustrates a diagrammatic representation of a machine in the exemplary form of a computer system 800 within which a set of instructions, for causing the machine to perform any one or more of the methodologies discussed herein, may be executed. In alternative implementations, the machine may be connected (e.g., networked) to other machines in a LAN, an intranet, an extranet, or the Internet. The machine may operate in the capacity of a server or a client machine in client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The exemplary computer system 800 includes a processing device (processor) 802, a main memory 804 (e.g., read-only memory (ROM), flash memory, dynamic random access memory (DRAM) such as synchronous DRAM (SDRAM) or Rambus DRAM (RDRAM), etc.), a static memory 806 (e.g., flash memory, static random access memory (SRAM), etc.), and a data storage device 818, which communicate with each other via a bus 808.

Processor 802 represents one or more general-purpose processing devices such as a microprocessor, central processing unit, or the like. More particularly, the processor 802 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor 802 may also be one or more special-purpose processing devices such as an application specific integrated circuit (ASIC), a field programmable gate array (FPGA), a digital signal processor (DSP), network processor, or the like. The processor 802 is configured to execute instructions 826 for performing the operations and steps discussed herein.

The computer system 800 may further include a network interface device 822. The computer system 800 also may include a video display unit 810 (e.g., a liquid crystal display (LCD), a cathode ray tube (CRT), or a touch screen), an alphanumeric input device 812 (e.g., a keyboard), a cursor control device 814 (e.g., a mouse), and a signal generation device 820 (e.g., a speaker).

The data storage device 818 may include a computer-readable storage medium 824 on which is stored one or more sets of instructions 826 (e.g., software) embodying any one or more of the methodologies or functions described herein. The instructions 826 may also reside, completely or at least partially, within the main memory 804 and/or within the processor 802 during execution thereof by the computer system 800, the main memory 804 and the processor 802 also constituting computer-readable storage media. The instructions 826 may further be transmitted or received over a network 874 via the network interface device 822.

In one implementation, the instructions 826 include instructions for a parameter monitoring component 212. While the computer-readable storage medium 824 is shown in an exemplary implementation to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the disclosure. The term “computer-readable storage medium” shall accordingly be taken to include, but not be limited to, solid-state memories, optical media, and magnetic media.

In the foregoing description, numerous details are set forth. It will be apparent, however, to one of ordinary skill in the art having the benefit of this disclosure, that the disclosure may be practiced without these specific details. In some instances, well-known structures and devices are shown in block diagram form, rather than in detail, in order to avoid obscuring the disclosure.

Some portions of the detailed description have been presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “segmenting”, “analyzing”, “determining”, “enabling”, “identifying,” “modifying” or the like, refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices.

The disclosure also relates to an apparatus for performing the operations herein. This apparatus may be specially constructed for the required purposes, or it may include a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions.

The words “example” or “exemplary” are used herein to mean serving as an example, instance, or illustration. Any aspect or design described herein as “example’ or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form.

Reference throughout this specification to “one embodiment” or “an embodiment” means that a particular feature, structure, or characteristic described in connection with the embodiment is included in at least one embodiment. Thus, the appearances of the phrase “in one embodiment” or “in an embodiment” in various places throughout this specification are not necessarily all referring to the same embodiment. In addition, the term “or” is intended to mean an inclusive “or” rather than an exclusive “or.”

The above description is intended to be illustrative and not restrictive. For example, the above-described examples (or one or more aspects thereof) may be used in combination with each other. Other implementations can be used, such as by one of ordinary skill in the art upon reviewing the above description. The abstract is provided to comply with 37 CFR 1.72(b) to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed implementation. Thus, the following claims are hereby incorporated into the Detailed Description, with each claim standing on its own as a separate implementation, and it is contemplated that such implementations can be combined with each other in various combinations or permutations. The scope of the implementations of the disclosure should be determined with reference to the appended claims, along with the full scope of equivalents to which such claims are entitled.

In the drawings, which are not necessarily drawn to scale, like numerals may describe similar components in different views. Like numerals having different letter suffixes may represent different instances of similar components. The drawings illustrate generally, by way of example, but not by way of limitation, various implementations discussed in the present document.

What is claimed is:

1. A therapy device comprising:
a plurality of sensors for measuring metrics comprising at least one of heart rate, range of motion, muscular activity, blood pressure, temperature, sweat or respiration of a patient, the measured metrics corresponding to an exercise;
a communication component operatively coupled to the plurality of sensors, the communication component to:
provide the measurements from the plurality of sensors to a computing device; and
receive feedback from the computing device based on comparison of the measured metrics from the plurality of sensors to a set of target metrics associated with the exercise; and
one or more lights operatively coupled to the communication component, the one or more lights to provide visual feedback associated with the exercise based on the received feedback from the computing device.

2. The therapy device of claim 1, wherein the one or more lights comprise light emitting diodes.

3. The therapy device of claim 2, further comprising:
a switching device operatively coupled to the plurality of lights, wherein the switching device allows for a selection of the visual feedback associated with the exercise to correspond to a particular measurement from the plurality of sensors.

4. The therapy device of claim 1, further comprising:
a liner to be disposed between skin of the patient and the plurality of sensors.

5. The therapy device of claim 4, wherein the liner comprises one or more portions comprised of at least one of conductive fabric or electrodes.

6. The therapy device of claim 1, wherein the plurality of sensors comprise:
at least one of an electrocardiogram sensor or a photoplethysmography sensor;
an inertia measurement unit sensor;
an electromyography sensor;
a Sphygmomanometer sensor;
a temperature sensor;
a sweat sensor; or
a respiratory sensor.

7. A method comprising:
receiving a content item corresponding to an exercise;
retrieving, from a storage device, a first stream of parameters associated with a plurality of timestamps for the content item;
aligning, by a processing device operatively coupled to the storage device, the content item with the first stream of parameters based on the plurality of timestamps;
determining a target value associated with at least one parameter of a stream of parameters during performance of the exercise;
capturing a second stream of parameters measured by a therapy device attached to a patient performing the exercise demonstrated in the content item;
determining that the at least one parameter of the second stream of parameters satisfies the target value; and
providing feedback indicating that the at least one parameter of the second stream of parameters satisfies the target value.

8. The method of claim 7, wherein the content item is associated with a content item identification and wherein the content item is stored in a cloud computing system.

9. The method of claim 8, wherein retrieving the first stream of parameters further comprises:
receiving the content item identification associated with the content item; and
identifying the first stream of parameters on the storage device based on the content item identification associated with the content item.

10. The method of claim 7, wherein providing the feedback indicating that the at least one parameter of the second stream of parameters satisfies the target value further comprises:
transmitting a signal to cause at least one of illumination of one or more lights associated with the therapy device or presentation of visual feedback associated with a computing device.

11. The method of claim 7, wherein receiving the content item further comprises:
recording a content item of the patient performing the exercise during a calibration session.

12. The method of claim 11, wherein the content item is stored in a local memory of a computing device.

13. The method of claim 7, wherein the target value is determined based on at least one of the first stream of parameters or a selection of the target value via a user interface of a computing device.

14. The method of claim 7, wherein receiving the content item corresponding to the exercise comprises:
selecting a previously recorded content item corresponding to the exercise from a library of content items.

15. The method of claim 7, wherein receiving the content item further comprises:
selecting one or more exercise segments from a plurality of exercise segments; and
generating the content item comprising the selected one or more exercise segments.

16. The method of claim 7, further comprising:
receiving an input to modify the target value for the at least one parameter of the stream of parameters; and
determining a second target value for the at least one parameter of the stream of parameters during a subsequent performance of the exercise.

17. A non-transitory computer-readable storage medium comprising instructions that, when executed by a processing device, cause the processing device to:
receive a content item corresponding to an exercise;
retrieve, from a storage device, a first stream of parameters associated with a plurality of timestamps for the content item;
align, by a processing device, the content item with the first stream of parameters based on the plurality of timestamps;
determine a target value associated with at least one parameter of a stream of parameters during performance of the exercise;
capture a second stream of parameters measured by a therapy device attached to a patient performing the exercise demonstrated in the content item;
determine that the at least one parameter of the second stream of parameters satisfies the target value; and
provide feedback indicating that the at least one of the second stream of parameters satisfies the target value.

18. The non-transitory computer-readable storage medium of claim 17, wherein the processing device is further to:
receive an input to modify the target value for the at least one parameter of the stream of parameters; and
determine a second target value for the at least one parameter of the stream of parameters during a subsequent performance of the exercise.

19. The non-transitory computer-readable storage medium of claim 17, wherein to provide the feedback indicating that the at least one parameter of the second stream of parameters satisfies the target value, the processing device is further to:
transmit a signal to cause at least one of illumination of one or more lights associated with the therapy device or presentation of visual feedback associated with a computing device.

20. The non-transitory computer-readable storage medium of claim 17, wherein the first stream of parameters is based on metric data acquired during a calibration session corresponding to the therapy device.
